(19) United States
(12) Patent Application Publication
(10) Pub. No.: US 2002/0165381 A1
(43) Pub. Date: Nov. 7, 2002

(54) Human androgen receptor variants

(76) Inventors: Isabelle Ahrens-Fath, Berlin (DE); Bernard Haendler, Berlin (DE)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 09/997,267

(22) Filed: Nov. 30, 2001

Related U.S. Application Data
(60) Provisional application No. 60/255,078, filed on Dec. 14, 2000.

(30) Foreign Application Priority Data
Nov. 30, 2000 (DE) 10061161.3

(51) Int. Cl. C07K 14/72; C07H 21/04; C12P 21/02; C12N 5/06

(52) U.S. Cl. 536/23.5; 530/350; 435/69.1; 435/320.1; 435/325

(57) ABSTRACT

Two new variants of the androgen receptor, AR42 and AR32, and their use are described.

This application claims the benefit of the filing date of U.S. Provisional Application Serial No. 60/255,078 filed Dec. 14, 2000.

The invention relates to two new variants of the androgen receptor and their use.

Androgens are the male sex hormones and are mainly produced in the testicle (Roy, A. K. et al., Vitam. Horm. 1999, 55, 309-352). They control the male sexual differentiation and are essential for spermatogenesis. In addition, androgens are responsible for the manifestation of the secondary sex characteristics and the sexual behavior pattern. Androgens also play an essential role in the development and reproduction of prostate and testicular cancer (Craft, N. and Sawyers, C. L., Cancer Metastasis Rev. 1998-99, 17, 421-427; Rajperts-De Meyts, E. and Skakkebaek, N. E., Eur. Urol. 1993, 23, 54-59).

Androgens act by binding to a specific nuclear receptor, the androgen receptor. The already known androgen receptor is a ligand-dependent transcription factor that has a ligand binding site, a DNA-binding site and several transactivation functions (Lindzey, J. et al., Vitam. Horm. 1994, 49, 383-432). The main transactivation function is found in the N-terminal half, which is coded by Exon 1 of the androgen receptor gene. If the ligand, an androgen, binds, the conformation of the receptor is changed. By this change in conformation, the receptor can form a dimer and can bind to a specific double-strand DNA sequence, which is called the steroid-response element. By interactions with co-activators and other transcription factors, the transcription of the target gene is activated (Lindzey, J. et al., Vitam. Horm. 1994, 49, 383-432).

Androgens play a role in hormone-dependent tumors. Thus, e.g., prostate cancer is treated with antiandrogens, which compete with the binding of natural androgens to the androgen receptor. In this connection, it is often shown that the antiandrogens are no longer effective after a certain treatment time (Crawford, E. D. et al., Urology 1999, 54, 1-7). As causes of this therapy resistance, mutations of the androgen receptor, which allow a stimulation by antiandrogens or by estrogens or glucocorticoids (Brinkmann, A. O. and Trapman, J., Nature Med. 2000, 6, 628-269) were postulated. These mutations occur relatively rarely, however. Another possible cause is an amplification of the androgen receptor gene, as described in about 28% of the androgen-resistant patients (Koivisto, P. et al., Cancer Res. 1997, 57, 314-319). This does not by any means explain all cases. For successful tumor therapy, it is therefore desirable to know another point of attack for the therapy.

This problem was solved by the preparation of two new variants of the androgen receptor.

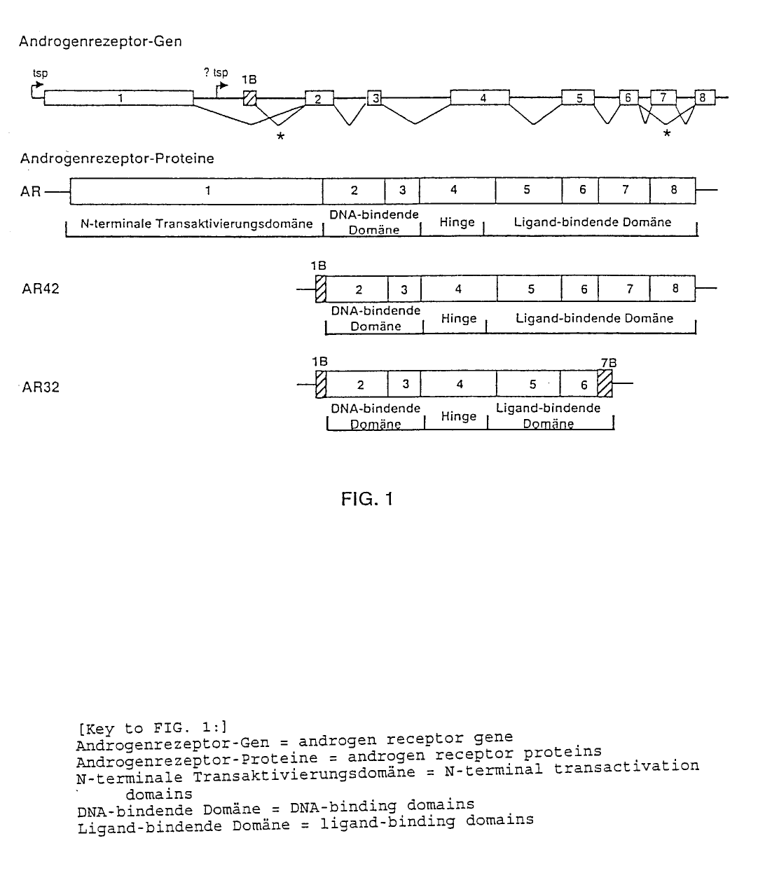

The first androgen receptor according to the invention with the amino acid sequence that is indicated in Seq ID NO 2 is named AR42 below, and the androgen receptor according to the invention with the amino acid sequence that is indicated in Seq ID NO 4 is named AR32. The already known androgen receptor (Lubahn, D. B. et al., Science 1988, 240, 327-330; Chang et al., Science 1988, 240, 324-326) has the designation AR below. The sequences of AR and AR42 are identical in the range of the DNA-binding domains, the so-called “hinge” domains and the ligand-binding domains (see Exons 2-8 in FIG. 1). They are different in the range of the N-terminus. While the AR here has a transactivation domain that is approximately 537 amino acids long, the AR42 here has only a 7-amino-acid range, whose sequence is different from the sequence of the transactivation domains of the AR. This 7-amino-acid range is not included in the genomic sequence in any previously known Exon; rather, it is a component of a DNA-range that previously was considered not translated.

The AR32 is distinguished from AR in the N-terminus and in the C-terminus (see Exons 1, 7 and 8 in FIG. 1). The AR32 has the same N-terminal sequence as AR42. It is distinguished from AR42 and from AR in the C-terminus. Its C-terminal sequence is shorter, and 10 amino acids are different compared to AR42 and AR.

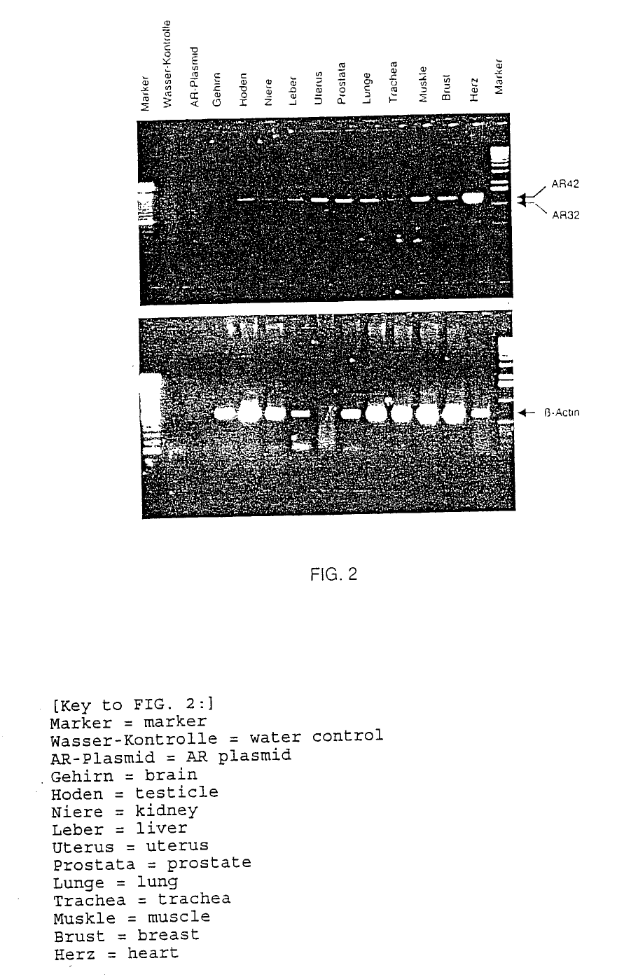

The AR42 and AR32 according to the invention are expressed in various tissues of healthy humans. AR42 is expressed especially strongly in the heart (FIG. 2).

The AR42 and AR32 according to the invention can bind androgen and other ligands. After binding to a ligand, AR42 and AR32 can form homodimers (AR42/AR42 or AR32/AR32) either below one another or with the AR heterodimers (AR42/AR or AR32/AR). The homodimers can bind to the steroid response element of AR; however, they cannot activate the transcription of the target genes, which activates the AR. AR42 and AR32 then act as repressors for the AR. By a heterodimer formation with the AR, the activity of the AR can be modulated. Whether this is inhibition or activation depends on the target genes. Activation of expression is carried out in target genes whose expression is blocked by an interaction of the AR with Ets transcription factors. These Ets transcription factors act as co-repressors for the AR by binding to its N-terminus (Schneikert, J. et al., J. Biol. Chem. 1996, 271, 23907-23913). They cannot bind to AR42 or AR32, however. As a result, the blocking is cancelled out, and the expression is stimulated.

The invention relates to nucleic acids that code for an androgen receptor, whereby they comprise

a. The nucleotide sequences that are shown in Seq ID NO 1 and/or 3,

b. a nucleotide sequence that corresponds to the sequence from a. within the scope of the degeneration of the genetic code, or

c. a nucleotide sequence that hybridizes with the sequences from a. and/or b. under stringent conditions.

The term “hybridization under stringent conditions” according to this invention is defined by Sambrook et al. (Molecular Cloning, A Laboratory Manual, Cold Spring Harbor Laboratory Press, 1989). A stringent hybridization exists, for example, if after washing for 1 hour with 1×SSC and 0.1% SDS at 50° C., preferably at 55° C., especially preferably at 62° C. and most preferably at 68° C., especially for 1 hour in 0.2×SSC and 0.1% SDS at 55° C., preferably at 62° C. and most preferably at 68° C., a hybridization signal is still observed. The nucleic acids, which hybridize under these conditions with the nucleic acid that is shown in Seq. ID NO 1 and/or 3 or a nucleotide sequence that corresponds to this sequence within the scope of the degeneration of the genetic code, are also the subject matter of this invention.

Nucleic acids can produce single- or double-strand DNA, e.g., cDNA, or RNA, e.g., mRNA, cRNA, or pre-mRNA.

Preferred are the nucleic acids that comprise a protein-coding section of the nucleic acid sequences that are shown in Seq ID NO 1 and/or 3. A protein-coding section of the sequence that is shown in Seq ID NO 1 is in the nucleotide range of 163 to 1329, and a protein-coding section of the sequence that is shown in Seq ID NO 3 is in the nucleotide range of NO 163 to NO 1047.

Subjects of the invention are also nucleic acids that code for a polypeptide with the amino acid sequence shown in Seq ID NO 2 and/or 4.

The nucleic acids according to the invention can be obtained from mammals, e.g., human cells, or from a cDNA library or a genomic library, which is obtained from, e.g., human cells. They can be isolated according to known techniques with use of short sections of the nucleic acid sequences that are shown in Seq ID NOS 1 and 3 as hybridization probes or amplification primers. Especially preferred are those sections that code for the peptide sequences that are shown in Seq ID NO 5 or 6.

In addition, the invention relates to polypeptides that are coded by a nucleic acid according to the invention. These polypeptides have the function of an androgen receptor. In addition, polypeptides that comprise the amino acid sequence that is shown in Seq ID NO 2 or 4 are subjects of the invention.

The polypeptides according to the invention can be recombinant polypeptides, natural, isolated polypeptides or synthetic polypeptides.

The invention also relates to peptides that comprise the sequence that is shown in Seq ID NO 5. The sequence that is shown in Seq ID NO 5 corresponds to the C-terminus (amino acid 285-294) of AR32.

The invention also relates to peptides that comprise the amino acid sequence that is shown in Seq ID NO 6. The amino acid sequence that is shown in Seq ID NO 6 corresponds to the N-terminus (amino acids 1-7) of AR42 and AR32.

The polypeptides according to the invention and the peptides according to the invention can be used for the production of antibodies. For the production of polyclonal antibodies, the peptides can be bonded to, e.g., KLH (keyhole limpet hemocyanin), and animals, e.g., rabbits, can be sprayed. They can also be used for the production of monoclonal antibodies. For antibody production, a peptide according to the invention or a mixture of several peptides according to the invention can be used. In this case, the production of the antibodies is carried out according to standard processes, as they are described in, e.g., Kohler, G. and Milstein, C., Nature 1975, 256, 495-497 and Nelson, P. N. et al., Mol. Pathol. 2000, 53, 111-117.

Subjects of the invention are also the antibodies that are directed against a polypeptide according to the invention or against a peptide according to the invention.

The antibodies according to the invention can be used for detection of the AR42 and AR32 according to the invention. This can be carried out by, e.g., immunohistochemistry. The detection of the polypeptides according to the invention in tumor tissue, especially in the tissue of prostate tumors, is preferred. It can be determined whether a hormone therapy resistance can be attributed to an altered expression of AR42 and/or AR32 according to the invention. The antibodies according to the invention can also be used in other immune tests, such as, e.g., an ELISA (enzyme linked immunosorbent assay) or in a radioimmuno test. Thus, the concentration of AR42 and AR32 according to the invention can be detected in tissue or cell extracts.

The detection of the expression of AR42 or AR32 can also be carried out via the detection of mRNA in the cells. The subject of the invention is therefore also the use of a probe with nucleic acid sequences that are complementary to the nucleic acid sequences that code for the peptides according to the invention for the production of a reagent for the detection of the presence of mRNA in tumor cells according to the invention. A probe is a short strand of DNA with at least 14 nucleotides. The probes according to the invention can be used in, e.g., a Northern Blot analysis. This method is described in, e.g., Sambrook, J. et al., 1989, Cold Spring Harbor Laboratory Press. Other methods for, detecting RNA are in-situ hybridization, RNAse protection assay or PCR.

In addition, subjects of the invention are vectors that contain at least one copy of a nucleic acid according to the invention. Vectors can be prokaryotic or eukaryotic vectors. Examples of vectors are pPRO (Clontech), PBAD (Invitrogen), pSG5 (Stragene), pCl (Promega), pIRES (Clontech), PBAC (Clontech), PMET (Invitrogen), pElueBac (Invitrogen). The nucleic acids according to the invention can be inserted into these vectors with the methods that are known to one skilled in the art. In connection with expression signals, such as, e.g., promoters and enhancers, the nucleic acids according to the invention are preferably found in the vector.

The invention also relates to cells that are transfixed with a nucleic acid sequence according to the invention or with a vector according to the invention. As cells, e.g., E. coli, yeast, Pichia, Sf9, COS, CV-1 or BHK can be used. Preferred are cells that are selected from the group that consists of PC-3 cells, LNCaP cells, CV-1 cells and Dunning cells. These cells can be used both for the production of AR42 and/or AR32 and for cell-based tests.

The subject of the invention is also the use of

a A nucleic acid according to the invention,

b. a polypeptide according to the invention,

c. a peptide with the amino acid sequence that is shown in Seq ID NO 5, or

d. a cell according to the invention

for identifying effectors of a polypeptide according to the invention. Effectors are substances that have an inhibitory or activating effect on the polypeptide according to the invention and that are able to influence the androgen receptor function of the polypeptides according to the invention.

In addition, the invention relates to a test system for detecting effectors of the polypeptides according to the invention, whereby

a. A reporter gene is expressed in the cells according to the invention, and

b. these cells, if they contain only a little or no polypeptide according to the invention, are transfixed in addition with a vector according to the invention,

c. the cells are cultivated in the presence or absence of the test substances and

d. the alteration of the expression of the reporter gene is measured.

The invention also relates to a test system for detecting test substances with antiandrogenic activity, whereby

a. A reporter gene is expressed in the cells according to the invention, and

b. these cells, if they contain only a little or no polypeptide according to the invention, are transfixed in addition with a vector according to the invention,

c. the cells are cultivated in the presence or absence of test substances with the simultaneous presence of an androgen, and

d. the alteration of the expression of the reporter gene is measured.

For a test system according to the invention, suitable cells, for example CV-1 cells, COS cells or cells that originate from the prostate, are transfixed in a stable or transient manner with a nucleic acid according to the invention or with portions thereof or with portions thereof in combination with a transactivation domain of other factors. Portions of a nucleic acid according to the invention can be, e.g., the ligand-binding domains, the transactivation domains and the DNA-binding domains. Transactivation domains of other factors can be, e.g., the ligand-binding domains, the transactivation domains and the DNA-binding domains of the AR or the progesterone receptor, the gal 4-transactivation domains or the VP16 transactivation domains. Reporter-plasmids can be co-transfixed. The latter contain one or more steroid-response elements, which produce inverted repeats of the TGTTCT sequence with a spacer of three base pairs. In addition, such response element direct repeats can be the TGTTCT sequence with a spacer of three to five base pairs. Deviations in the TGTTCT sequence, as described in Natural Response Elements, are possible (Kokontis, J. M. and Liao, S., Vitam. Horm. 1999, 55, 219-307). A minimal promoter (Schenborn, E. and Groskreutz, D., Mol. Biotechnol. 1999, 13, 29-44) and a heterologous reporter gene are downstream, in operative linkage. Reporter plasmids can also contain a promoter or promoter portions of known androgen-regulated genes. Genes that are androgen-dependent in the prostate are preferably expressed. Examples of this are the PSA, probasin and C3(l)-gene. Reporter genes can be, e.g., the luciferase gene, the chloramphenicol acetyltransferase gene, urokinase gene, green fluorescence protein gene and β-galactosidase gene. The test substances are preferably selected from the group of androgen derivatives. These test systems, however, can also be used to screen large substance libraries. For the test system for the detection of test substances with antiandrogenic activity, e.g., R1881, testosterone, dihydrotestosterone and testosterone derivatives can be used as androgens in step c.

Those substances are preferred that alter the expression of a reporter gene in a test system according to the invention but are not effectors of the AR. To determine whether the substances are effectors of AR, a test system can be used that is built up analogously to the test system according to the invention, whereby the cells are transfixed with a vector that contains the nucleic acid of AR instead of with a vector according to the invention.

By heterodimer formation of the polypeptides according to the invention with the AR, effectors that activate the polypeptides according to the invention but not the AR result in inhibition of the AR. This inhibitory action can be determined with a test system that is described in Example 5. Inhibition of the AR is desirable in all androgen-dependent diseases, e.g., for treatment of prostate tumors and also in male birth control. In male birth control, e.g., the expression of genes that are necessary for the formation of mature sperm can be inhibited by inhibition of the AR.

In addition, genes can be identified that are regulated selectively by homodimers or heterodimers of the polypeptides according to the invention. These genes can be identified by specific knock-out and knock-in experiments.

The invention also provides a process for the preparation of pharmaceutically active substances, whereby

a. The substances to be tested are brought into contact with a test system according to the invention,

b. the action of the substances on the test system is measured in comparison to the controls, and

c. a substance is identified that shows a modulation of the expression of the heterologous polypeptide in step b.

The invention also relates to a process for the preparation of a pharmaceutical agent, whereby

a. The substances to be tested are brought into contact with a test system according to the invention,

b. the action of the substances on the test system optionally is measured in comparison to the controls,

c. a substance is identified that shows a modulation of the expression of the heterologous polypeptide in step b.,

d. and the substance that is identified in step c is mixed with the formulation substances that are commonly used in pharmaceutics.

The invention also provides a process for the preparation of a pharmaceutical agent, whereby

a. Substances are brought into contact with a test system according to the invention,

b. the action of the substances on the test system in comparison to controls is measured,

c. a substance that shows a modulation of the expression of the heterologous polypeptide in step b. is identified,

d. the substance that is identified in step c. is optionally optimized, and

e. this optionally optimized substance is mixed with formulation substances that are commonly used in pharmaceutics.

Preferred are substances that increase at least 10-fold or inhibit the reporter gene activity in the test systems according to the invention. A substance that is identified by a process according to the invention can optionally be optimized relative to metabolic stability, activity in a test system according to the invention and/or bio-availability. To this end, methods that are common in chemistry can be used.

The preferred preparations consist in a form of dispensing that is suitable for oral, enteral or parenteral administration. Such forms for dispensing are, for example, tablets, film tablets, coated tablets, pills, capsules, powder or depot forms as well as suppositories. Corresponding tablets can be obtained, for example, by mixing active ingredient with known adjuvants, for example inert diluents such as dextrose, sugar, sorbitol, mannitol, polyvinylpyrrolidone, explosives such as corn starch or alginic acid, binders such as starch or gelatin, lubricants such as carboxypolymethylene, carboxymethyl cellulose, cellulose acetate phthalate or polyvinyl acetate. The tablets can also consist of several layers.

Coated tablets can be produced accordingly by coating nuclei that are produced analogously to the tablets with agents that are commonly used in coated tablet coatings, for example, polyvinylpyrrolidone or shellac, gum arabic, talc, titanium oxide or sugar. In this case, the coated tablet shell can also consist of several layers, whereby the adjuvants that are mentioned above in the tablets can be used. Capsules that contain active ingredients can be produced, for example, by the active ingredient being mixed with an inert vehicle such as lactose or sorbitol and being encapsulated in gelatin capsules. The substances according to the invention can also be used in suitable solutions such as, for example, physiological common salt solution, as an infusion or injection solution. For parenteral administration, especially oily solutions, such as, for example solutions in sesame oil, castor oil and cottonseed oil, are suitable. To increase the solubility, solubilizers, such as, for example, benzyl benzoate or benzyl alcohol, can be added. It is also possible to incorporate the substances that are obtainable and that are obtained into a transdermal system via the process according to the invention and thus to administer them transdermally.

The pharmaceutical agent according to the invention can be used for the production of a medication for the treatment of androgen-dependent diseases. Such diseases can be, e.g., prostate cancer or testicular tumors.

The pharmaceutical agent according to the invention can be used for the production of a medication for male birth control.

Androgen-dependent diseases can be influenced, on the one hand, as described above by effectors of the polypeptides according to the invention, but also, on the other hand, by an alteration of the concentration of the polypeptides according to the invention in the affected tissues. For this purpose, either a nucleic acid according to the invention with the aid of a vector that is commonly used in gene therapy or a polypeptide according to the invention can be brought into the tissue. In gene therapy, a vector that contains a nucleic acid according to the invention is designed and administered. Examples are vectors that are derived from the adenovirus, andenovirus-associated virus, Herpes simplex virus or SV40. The gene therapy can be performed according to a protocol as described by Gomez-Navarro, J. et al. (Eur. J. Cancer 1999, 35, 867-885). The administration can be carried out locally, i.e., directly into the affected tissue, such as, e.g., the prostate tumor, or systemically, i.e., via the blood flow. This results in an elevated expression of the polypeptide according to the invention.

The administration of a polypeptide according to the invention can be carried out in the form of a fusion polypeptide. The polypeptide according to the invention is preferably transported to the desired tissue, e.g., to the prostate tumor tissue, by the fused polypeptide, e.g., EGF or transferrin.

### BRIEF DESCRIPTION OF THE DRAWINGS

Various other features and attendant advantages of the present invention will be more fully appreciated as the same becomes better understood when considered in conjunction with the accompanying drawings, in which like reference characters designate the same or similar parts throughout the several views, and wherein:

### DESCRIPTION OF THE FIGURES

FIG. 1 shows the Intron-Exon structure of the AR gene and the domain structure of AR, AR42 and AR32. In the gene, the known transcription start (tsp) in the promoter and the putative 2nd transcription start (?tsp) from the Exon 1B were indicated with arrows. The new Exon 1B is cross-hatched. Stars indicate splicing events that specifically result in the generation of mRNA for AR42 and AR32. In the protein, the various domains were shown. The new regions that are added to AR42 and AR32 by the alternative splicing of the AR Gene are cross-hatched.

FIG. 2 shows the tissue distribution of AR42 and AR32 MRNA. A sense primer, which was directed against the specific AR42 and AR32 Exon, and an antisense primer, which was directed against the common C-terminal region, were used for the PCR amplification. Total-RNA was obtained from various human tissues and transcribed with a reverse transcriptase. This First-Strand cDNA was used as a template. The PCR products were separated on an agarose gel and stained with ethidium bromide. The AR42 cDNA can be detected as a strong band, and the AR32 cDNa can be detected as a very weak band on the gel.

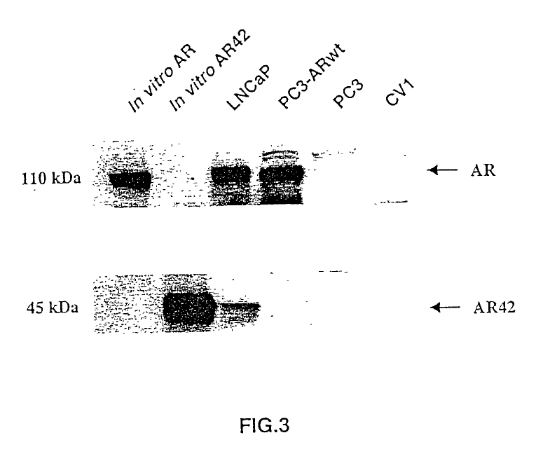

FIG. 3 shows the expression of the AR42 protein in LNCaP cells. An antibody that was directed against the ligand-binding domains of AR was used for the Western Blot analysis. Entire cell extracts from various cell lines (LNCaP, PC-3ARwt, PC-3, CV-1) were applied to an 8% tris-glycine gel. In vitro-translated AR and AR42 proteins were applied as controls. The AR42 protein was detected only in LNCaP cells. The 110 kDa AR protein was detected in LNCaP and in PC3-ARwt. The PC-3ARwt cell line was obtained by transfixing PC-3 cells with a plasmid that contains AR.

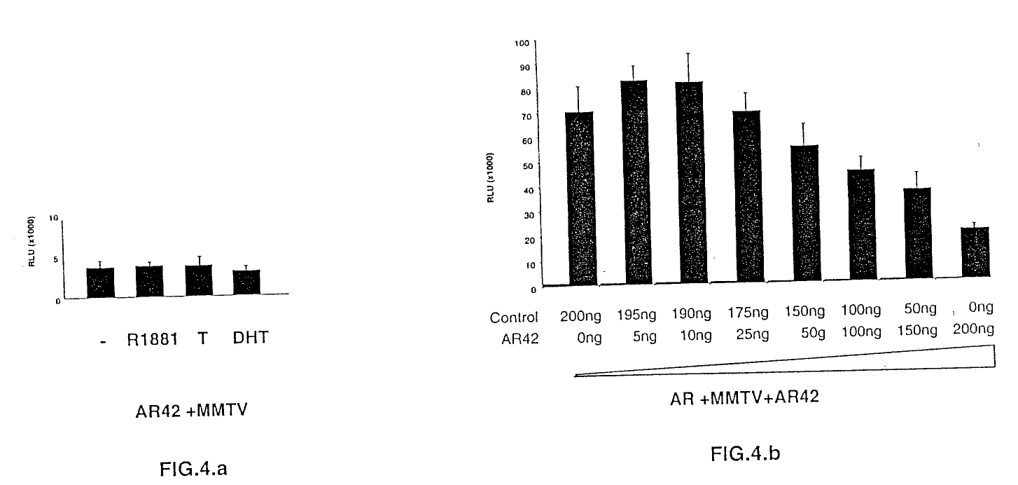

FIG. 4a shows that AR42 does not have any transactivating function in PC-3 cells. 100 ng of pSG5-AR42 was co-transfixed in PC-3 together with 100 ng of a reporter plasmid, which contains the MMTV promoter. These cells were then treated with various androgens (R1881: metribolone; T: testosterone; DHT: 5α-dihydrotestosterone) with a final concentration of 10⁻⁷ M.

FIG. 4b shows the transrepressing activity of AR42. 10 ng of pSG5-AR and different amounts of pSG5-AR42 were co-transfixed in PC-3 cells. In addition, 100 ng of a reporter plasmid, which contains the MMTV promoter, was transfixed. The reporter gene activity was measured after treatment with R1881 (10⁻⁹ M) Increasing amounts of transfixed pSG5-AR42 inhibited the transactivating effects of stimulated AR.

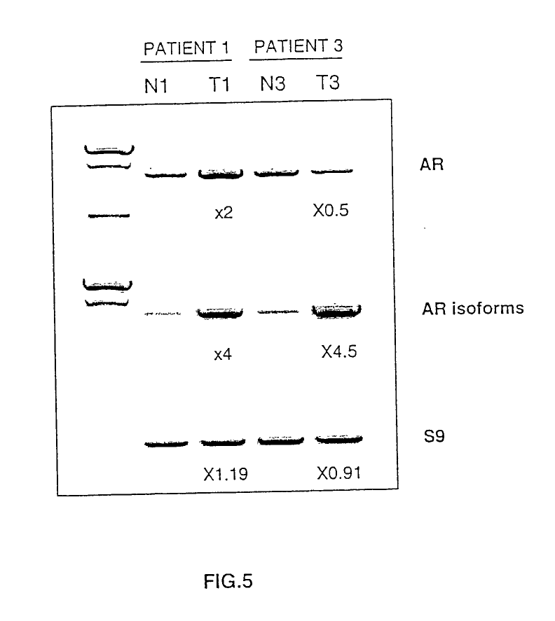

FIG. 5 shows the expression of AR42 MRNA in prostate tumor tissue. Whole prostate RNA was obtained from normal (N) or tumor (T) tissue of two prostate cancer patients and converted with a reverse transcriptase. A sense primer, which was directed against the specific AR42 and AR32 Exon, and an antisense primer, which was directed against the common AR C-terminal region, were used for PCR amplification. AR and S9 DNA fragments were reamplified in parallel. The S9 DNA amounts are used as an internal standard. The results show that AR42-RNA in both prostate tumor tissues is more strongly expressed than in normal tissue. The AR transcript amounts do not have any comparable changes. The designations ×2, ×0.5, etc. indicate by which factor the respective band in the tumor tissue is stronger compared to the normal tissue.

Without further elaboration, it is believed that one skilled in the art can, using the preceding description, utilize the present invention to its fullest extent. The following preferred specific embodiments are, therefore, to be construed as merely illustrative, and not limitative of the remainder of the disclosure in any way whatsoever.

In the foregoing and in the following examples, all temperatures are set forth uncorrected in degrees Celsius and, all parts and percentages are by weight, unless otherwise indicated.

The entire disclosure[s] of all applications, patents and publications, cited above, and of corresponding German application No. 10061161.3, filed Nov. 30, 2001, and U.S. Provisional Application Serial No. 60/255,078, filed Dec. 14, 2000, are hereby incorporated by reference.

### EXAMPLES

The molecular-biological methods that are used in the Examples, such as, e.g., polymerase chain reaction (PCR), production of cDNA, cloning of DNA, sequencing of DNA, were performed as described in known textbooks, such as, for example, in Molecular Cloning, A Laboratory Manual (Sambrook, J. et al., 1989, Cold Spring Harbor Laboratory Press).

### Example 1

Identification and Cloning of AR42 and AR32

Starting material was 1 μg of total-RNA from human placenta, which was converted by means of the SMART RACE amplification kit (Clontech) into cDNA. For PCR amplification, the Advantage-2 PCR kit (Clontech) was used together with an antisense primer (5′-CAGATTACCAAGCTTCAGCTTCCG-3′), which is directed against the Hinge region of the human androgen receptor and uses a sense 5′-Smart II primer. The reaction conditions were: 5 seconds at 94° C., 3 minutes at 72° C. (5 cycles) ; 5 seconds at 94° C., 10 seconds at 70° C., 3 minutes at 72° C. (5 cycles) ; 5 seconds at 94° C., 10 seconds at 68° C., 3 minutes at 72° C. (27 cycles). To this end, a fragment of about 500 base pairs was amplified, purified on agarose gel, cloned in the PCR-TOPO plasmid (Invitrogen) and sequenced. The DNA sequence showed that the complete DNA-binding domain of the androgen receptor was present (corresponds to Exons 2 and 3 in the androgen receptor gene). In addition, a new sequence was linked immediately before Exon 2. This section, which can be designated as Exon 1B, contains about 160 base pairs of the untranslated range and a short, new sequence that codes for 7 amino acids. To isolate the complete cDNA, the sense primer 5′-ACAGGGAACCAGGGAAACGAATGCAGAGTGCTCCTGACATTGCCTGT-3′ (final concentration 0.2 μm) and 5′-GACAGGGAACCAGGGAAACGAATG-3′ (final concentration 1 μm), which originate from the new Exon 1B-range, and an antisense primer (5′-TCACTGGGTGTGGAAATAGATGGGCTTGA-3′), which codes for the C-terminal end of the known AR, were synthesized. The specified conditions for the SMART-PCR were used. It thus is possible to amplify and to clone a fragment of about 1200 base pairs from the same cDNA placenta. After DNA sequencing, it turned out that there were two different fragments, as shown in Seq ID NO 1 and NO 3. In both cases, the new portion that corresponds to Example 1B was present. The difference between AR42 and AR32 was in the C-terminal range, since in AR32, the region that is coded by Exon 7 was missing. A search in the genomic data bases showed that the new Exon 1B range is in the middle of the first Intron of the androgen receptor gene. An analysis of the gene section that precedes it shows that a second promoter of the androgen receptor gene is possibly in this region. This section contains putative initiator regions that are used in the detection by the basal transcription machinery, as well as several putative steroid hormone-responsive elements.

### Example 2

Tissue Distribution of AR42 and AR32

The tissue distribution was determined by semi-quantitative PCR. The primers, which were used for the isolation of complete AR42 and AR32-cDNA sequences (Example 1), were also used here. In the control, specific primers for beta-actin were used (sense primer: 5′-TGACGGGGTCACCCACACTGTGCCCATCTA-3′; antisense primer: 5′-CTAGAAGCATTTGCGGTGGACGATGGAGGG-3′). Total-RNA from the following human tissues was used: brain, testicle, kidney, liver, uterus, prostate, lung, trachea, muscle, breast, heart. After transcription in first-strand cDNA (Stratagene), a PCR analysis was performed with the Advantage-2 PCR kit (Clontech). The reaction conditions were: 5 seconds at 94° C., 3 minutes at 72° C. (5 cycles) ; 5 seconds at 94° C., 10 seconds at 70° C., 3 minutes at 72° C. (5 cycles) ; 5 seconds at 94° C., 10 seconds at 68° C., 3 minutes at 72° C. (20 cycles). The amplification products were separated on a 1% agarose gel and stained with ethidium bromide. The results showed that AR42 RNA was expressed most often in the heart, muscle, uterus and in the prostate. The AR32 RNA amounts were generally low and did not show any significant differences between tissues.

### Example 3

Expression of AR42 and AR32

For the expression of the total AR42 or AR32, the coding range in the baculovirus expression vector pBlueBac 4.5 (Invitrogen) was introduced. To simplify detection and purification, a fusion was carried out with an His tag. After co-transfection of insect cells with the Bac-N-Blue DNA, recombinant viruses were produced that were identified by a PCR process. A phage stock was then applied and used in larger amounts for additional transfections and production of AR42 or AR32. The purification of the His-tagged proteins was carried out via a nickel affinity column.

### Example 4

Test System for Finding Effectors

A vector for the transient expression of AR42 or AR32 is built in the pSG5 plasmid (Stratagene). This vector is transfixed in CV-1 or PC-3 cells. Parallel to this, a reporter plasmid that contains one or more copies of a steroid response element or a selective androgen response element, coupled to a luciferase reporter gene, is cotransfixed. To find specific effectors of AR42 or AR32, a high-throughput screening of substance banks is to be performed. Substances that trigger the activity of the reporter gene at a concentration of 10⁻⁶ M or less are further processed. The search for receptor antagonists is performed in the presence of 10⁻⁹ M androgen, e.g., R1881. Substances are selected that at least divide in half the androgen induction at a concentration of 10⁻⁶ M or less.

### Example 5

Transrepressing Activity of AR42 and AR32

A vector for the transient expression of AR42, AR32 or AR is built in the pSG5 plasmid (Stratagene). A constant amount of pSG5-AR and varying amounts of pSG5-AR42 or pSG5-AR32 are transfixed in CV-1 cells. In the control, a pSGS plasmid, which contains an irrelevant cDNA of similar length, is cotransfixed with the pSG5-AR. In addition, a reporter plasmid, which contains one or more copies of a steroid-response element or a selective androgen-response element, coupled to a luciferase reporter gene, is cotransfixed. After treatment with an androgen, an increase in the reporter gene activity is measured. Increasing amounts of transfixed pSG5-AR42 or pSG5-AR32 inhibit these transactivating effects of stimulated AR.

### Example 6

Detection of the Expression of the AR42 Protein

AR42 or AR was translated in vitro with the TNT T7 Quick Coupled Transcription/Translation System (Promega). To this end, pSG5-AR42 or PSG5-AR was incubated with rabbit reticulocyte lysate mix at 30° C. for 90 minutes. Part of the feedstock (1/25) or the entire cell extract (40 ng) was separated on an 8% tris-glycine gel and transferred in Towbin buffer onto a nitrocellulose membrane. The transfer membrane was blocked in PBS-Tween buffer with 5% milk. The primary antibody was a polyclonal antibody from rabbits, which was directed against the ligand-binding domain of AR. This antibody was diluted 1/500 in PBS-Tween buffer with 3% milk. For detection, the Amersham ECL kit was used.

### Example 7

RNA Expression in Prostate Tumors

The expression of AR42 and AR32 MRNA in prostate tumors was determined by semi-quantitative PCR. The primers described in Example 2 were used. In the control, specific primers were used for human ribosomal protein S9 (sense primer: 5′-GATGAGAAGGACCCACGGCGTCTGTTCG-3′; antisense primer: 5′-GAGACAATCCAGCAGCCCAGGAGGGACA-3′) and for AR (sense primer: 5′-CCCTGGATGGATAGCTACTCCGGACCTTACGGGGACATGCGT-3′; antisense primer: 5′-TCACTGGGTGTGGAAATAGATGGGCTTGA-3′). Whole RNA from normal prostate tissue or from prostate tumors was analyzed as in Example 2. The optical density of the bands was measured with the Gel Doc system and the Quantity One software of Biorad.

The preceding examples can be repeated with similar success by substituting the generically or specifically described reactants and/or operating conditions of this invention for those used in the preceding examples.

From the foregoing description, one skilled in the art can easily ascertain the essential characteristics of this invention and, without departing from the spirit and scope thereof, can make various changes and modifications of the invention to adapt it to various usages and conditions.

1. Nucleic acid that codes for an androgen receptor, characterized in that it comprises
a. The nucleotide sequences that are shown in Seq ID NO 1 and/or 3,
b. a nucleotide sequence that corresponds to the sequence from a. within the scope of the degeneration of the genetic code, or
c. a nucleotide sequence that hybridizes with the sequences from a. and/or b. under stringent conditions.

2. Nucleic acid according to claim 1, wherein it comprises a protein-coding section of the nucleic acid sequences that are shown in Seq ID NO 1 and/or 3.

3. Nucleic acid, wherein it codes for a polypeptide with the amino acid sequence that is shown in Seq ID NO 2 and/or 4.

4. Polypeptide, wherein it is coded by a nucleic acid according to one of claims 1-3.

5. Polypeptide, wherein it comprises the amino acid sequence that is shown in Seq ID NO 2 or 4.

6. Peptide, wherein it comprises the sequence that is shown in Seq. ID NO 5.

7. Peptide, wherein it comprises the amino acid sequence that is shown in Seq. ID NO 6.

8. Use of a polypeptide according to claim 4 or 5 or a peptide according to claim 6 and/or 7 for the production of antibodies.

9. Antibodies against a polypeptide according to one of claims 4 or 5 or against a peptide according to claim 6 or 7.

10. Use of an antibody according to claim 9 for detection of a polypeptide according to claim 4 or 5 in the tumor tissue.

11. Use of a probe with nucleic acid sequences that are complementary to the nucleic acid sequences, that code for the peptides according to claims 6 or 7, for the production of a reagent for detecting the presence of mRNA in tumor cells according to one of claims 1 to 3.

12. Vector, wherein it contains at least one copy of a nucleic acid according to one of claims 1-3.

13. Cell, wherein it is transfixed with a nucleic acid according to one of claims 1-3 or with a vector according to claim 12.

14. Cell according to claim 13, wherein it is selected from the group that consists of PC-3 cells, LNCaP cells, CV-1 cells, CV-1 cells and Dunning cells.

15. Use of a cell according to claim 13 or 14 for the expression of nucleic acid according to one of claims 1-3.

16. Use of
a. A nucleic acid according to one of claims 1 to 3,
b. a polypeptide according to claim 4 or 5,
c. a peptide with the amino acid sequence that is shown in Seq ID NO 5 or
d. a cell according to claim 13 or 14 to identify effectors of a polypeptide according to claim 4 or 5.

17. Test system for detecting effectors of the polypeptides according to the invention, whereby
a. A reporter gene is expressed in a cell according to claim 13 or 14, and
b. this cell, if it contains only a little or no polypeptide according to claim 4 or 5, is transfixed in addition with a vector according to claim 12,
c. the cells are cultivated in the presence or absence of the test substances and
d. the alteration of the expression of the reporter gene is measured.

18. Test system for detecting test substances with antiandrogenic activity, whereby
a. A reporter gene is expressed in a cell according to claim 13 or 14, and
b. this cell, if it contains only a little or no polypeptide according to claim 4, is transfixed in addition with a vector according to claim 12,
c. the cell is cultivated in the presence or absence of test substances with the simultaneous presence of an androgen, and
d. the alteration of the expression of the reporter gene is measured.

19. Process for the preparation of pharmaceutically active substances, whereby
a. Substances are brought into contact with a test system according to claim 17 or 18,
b. the action of the substances on the test system is measured in comparison to the controls, and
c. a substance is identified that shows a modulation of the expression of the heterologous polypeptide in step b.

20. Process for the preparation of a pharmaceutical agent, whereby
a. Substances are brought into contact with a test system according to claim 17 or 18,
b. the action of the substances on the test system in comparison to controls is measured,
c. a substance that shows a modulation of the expression of the heterologous polypeptide in step b. is identified,
d. and the substance that is identified in step c. is mixed with formulation substances that are commonly used in pharmaceutics.

21. Use of a substance that is prepared according to claim 19 or a pharmaceutical agent that is prepared according to claim 20 for the production of a medication for the treatment of androgen-dependent diseases.

22. Use of a substance that is prepared according to claim 19 or a pharmaceutical agent that is prepared according to claim 20 for the production of a medication for male birth control.

23. Use of a nucleic acid according to one of claims 1-3 in the gene therapy of androgen-dependent diseases.


SEQUENCE LISTING (15 sequences)

SEQ ID NO: 1 - DNA, 1329 bp, Homo sapiens
gctgcgagcagagaggggttcctcggaggtcatctgttccatcttcttgcctatgcaaatgcctgcctgaagctgctggaggctggctttgtaccggactttgtacagggaaccagggaaacgaatgcagagtgctcctgacattgcctgtcactttttcccatgatactctggcttcacagtttggagactgccagggaccatgttttgcccattgactattactttccaccccagaagacctgcctgatctgtggagatgaagcttctgggtgtcactatggagctctcacatgtggaagctgcaaggtcttcttcaaaagagccgctgaagggaaacagaagtacctgtgcgccagcagaaatgattgcactattgataaattccgaaggaaaaattgtccatcttgtcgtcttcggaaatgttatgaagcagggatgactctgggagcccggaagctgaagaaacttggtaatctgaaactacaggaggaaggagaggcttccagcaccaccagccccactgaggagacaacccagaagctgacagtgtcacacattgaaggctatgaatgtcagcccatctttctgaatgtcctggaagccattgagccaggtgtagtgtgtgctggacacgacaacaaccagcccgactcctttgcagccttgctctctagcctcaatgaactgggagagagacagcttgtacacgtggtcaagtgggccaaggccttgcctggcttccgcaacttacacgtggacgaccagatggctgtcattcagtactcctggatggggctcatggtgtttgccatgggctggcgatccttcaccaatgtcaactccaggatgctctacttcgcccctgatctggttttcaatgagtaccgcatgcacaagtcccggatgtacagccagtgtgtccgaatgaggcacctctctcaagagtttggatggctccaaatcaccccccaggaattcctgtgcatgaaagcactgctactcttcagcattattccagtggatgggctgaaaaatcaaaaattctttgatgaacttcgaatgaactacatcaaggaactcgatcgtatcattgcatgcaaaagaaaaaatcccacatcctgctcaagacgcttctaccagctcaccaagctcctggactccgtgcagcctattgcgagagagctgcatcagttcacttttgacctgctaatcaagtcacacatggtgagcgtggactttccggaaatgatggcagagatcatctctgtgcaagtgcccaagatcctttctgggaaagtcaagcccatctatttccacacccagtga

SEQ ID NO: 2 - protein, 388 aa, Homo sapiens
MILWLHSLETARDHVLPIDYYFPPQKTCLICGDEASGCHYGALTCGSCKVFFKRAAEGKQKYLCASRNDCTIDKFRRKNCPSCRLRKCYEAGMTLGARKLKKLGNLKLQEEGEASSTTSPTEETTQKLTVSHIEGYECQPIFLNVLEAIEPGVVCAGHDNNQPDSFAALLSSLNELGERQLVHVVKWAKALPGFRNLHVDDQMAVIQYSWMGLMVFAMGWRSFTNVNSRMLYFAPDLVFNEYRMHKSRMYSQCVRMRHLSQEFGWLQITPQEFLCMKALLLFSIIPVDGLKNQKFFDELRMNYIKELDRIIACKRKNPTSCSRRFYQLTKLLDSVQPIARELHQFTFDLLIKSHMVSVDFPEMMAEIISVQVPKILSGKVKPIYFHTQ

SEQ ID NO: 3 - DNA, 1171 bp, Homo sapiens
gctgcgagcagagaggggttcctcggaggtcatctgttccatcttcttgcctatgcaaatgcctgcctgaagctgctggaggctggctttgtaccggactttgtacagggaaccagggaaacgaatgcagagtgctcctgacattgcctgtcactttttcccatgatactctggcttcacagtttggagactgccagggaccatgttttgcccattgactattactttccaccccagaagacctgcctgatctgtggagatgaagcttctgggtgtcactatggagctctcacatgtggaagctgcaaggtcttcttcaaaagagccgctgaagggaaacagaagtacctgtgcgccagcagaaatgattgcactattgataaattccgaaggaaaaattgtccatcttgtcgtcttcggaaatgttatgaagcagggatgactctgggagcccggaagctgaagaaacttggtaatctgaaactacaggaggaaggagaggcttccagcaccaccagccccactgaggagacaacccagaagctgacagtgtcacacattgaaggctatgaatgtcagcccatctttctgaatgtcctggaagccattgagccaggtgtagtgtgtgctggacacgacaacaaccagcccgactcctttgcagccttgctctctagcctcaatgaactgggagagagacagcttgtacacgtggtcaagtgggccaaggccttgcctggcttccgcaacttacacgtggacgaccagatggctgtcattcagtactcctggatggggctcatggtgtttgccatgggctggcgatccttcaccaatgtcaactccaggatgctctacttcgcccctgacctggttttcaatgagtaccgcatgcacaagtcccggatgtacagccagtgtgtccgaatgaggcacctctctcaagagtttggatggctccaaatcaccccccaggaattcctgtgcatgaaagcactgctactcttcagcattaattgcgagagagctgcatcagttcacttttgacctgctaatcaagtcacacatggtgagcgtggactttccggaaatgatggcagagatcatctctgtgcaagtgcccaagatcctttctgggaaagtcaagcccatctatttccacacccagtga

SEQ ID NO: 4 - protein, 294 aa, Homo sapiens
MILWLHSLETARDHVLPIDYYFPPQKTCLICGDEASGCHYGALTCGSCKVFFKRAAEGKQKYLCASRNDCTIDKFRRKNCPSCRLRKCYEAGMTLGARKLKKLGNLKLQEEGEASSTTSPTEETTQKLTVSHIEGYECQPIFLNVLEAIEPGVVCAGHDNNQPDSFAALLSSLNELGERQLVHVVKWAKALPGFRNLHVDDQMAVIQYSWMGLMVFAMGWRSFTNVNSRMLYFAPDLVFNEYRMHKSRMYSQCVRMRHLSQEFGWLQITPQEFLCMKALLLFSINCERAASVHF

SEQ ID NO: 5 - protein, 10 aa, Homo sapiens
NCERAASVHF

SEQ ID NO: 6 - protein, 7 aa, Homo sapiens
MILWLHS

SEQ ID NO: 7 - DNA, 24 bp, Artificial Sequence
  Description of Artificial Sequence Primer
cagattaccaagcttcagcttccg

SEQ ID NO: 8 - DNA, 47 bp, Artificial Sequence
  Description of Artificial Sequence Primer
acagggaaccagggaaacgaatgcagagtgctcctgacattgcctgt

SEQ ID NO: 9 - DNA, 24 bp, Artificial Sequence
  Description of Artificial Sequence Primer
gacagggaaccagggaaacgaatg

SEQ ID NO: 10 - DNA, 29 bp, Artificial Sequence
  Description of Artificial Sequence Primer
tcactgggtgtggaaatagatgggcttga

SEQ ID NO: 11 - DNA, 30 bp, Artificial Sequence
  Description of Artificial Sequence Primer
tgacggggtcacccacactgtgcccatcta

SEQ ID NO: 12 - DNA, 30 bp, Artificial Sequence
  Description of Artificial Sequence Primer
ctagaagcatttgcggtggacgatggaggg

SEQ ID NO: 13 - DNA, 28 bp, Artificial Sequence
  Description of Artificial Sequence Primer
gatgagaaggacccacggcgtctgttcg

SEQ ID NO: 14 - DNA, 28 bp, Artificial Sequence
  Description of Artificial Sequence Primer
gagacaatccagcagcccaggagggaca

SEQ ID NO: 15 - DNA, 42 bp, Artificial Sequence
  Description of Artificial Sequence Primer
ccctggatggatagctactccggaccttacggggacatgcgt